(19) United States
(12) Patent Application Publication
(10) Pub. No.: US 2008/0287985 A1
(43) Pub. Date: Nov. 20, 2008

(54) Disposable surgical instrument having a locking device

(76) Inventors: James K. Patterson, Memphis, TN (US); Dimitri K. Protopsaltis, Memphis, TN (US)

Correspondence Address: WYATT, TARRANT & COMBS, LLP, 1715 AARON BRENNER DRIVE, SUITE 800, MEMPHIS, TN, 38120-4367, US

(21) Appl. No.: 11/804,548

(22) Filed: May 18, 2007

(51) Int. Cl. A61B 17/28

(52) U.S. Cl. 606/208

(57) ABSTRACT

The present invention discloses a surgical instrument having a locking device which locks the instrument in a closed position while bearing a load. The surgical instrument, which may function as forceps, or a tanaculum, is constructed of a disposable material in order to provide a single use medical instrument. By constructing the invention of a disposable material, the unique shape of the disclosed embodiment allows movement of the arms in order to allow tip penetration along the path of least resistance while also minimizing tissue damage and bleeding. The locking device described may further be used on other instruments.

### CROSS REFERENCE TO RELATED APPLICATIONS

Not applicable

### STATEMENT REGARDING FEDERALLY SPONSORED RESEARCH OR DEVELOPMENT

Not applicable

### REFERENCE TO A “MICROFICHE APPENDIX”

Not applicable

### FIELD OF THE INVENTION

The present invention relates to the field of surgical instruments having locking devices.

### BACKGROUND OF THE INVENTION

During certain gynecological procedures, it is necessary to grasp certain tissue. Surgical instruments, such as a tanaculum, have two pinching, or piercing, type tips located on arms, in opposed positions, such that the instrument may be closed in order to grip a certain tissue. Such surgical instruments have previously been made of a heavy set and durable stainless steel which provides durability of the instrument during sterilization, such as autoclaving, as well as the pinching, or piercing, function of grasping the tissue. Such surgical instruments exert damage to the tissue and promote tissue bleeding when being used. The stainless steel material is not forgiving in terms of penetrating tissue, or tearing tissue if the surgical instrument is pulled away from the tissue while in a closed position. It is an object of the present invention to resolve the problems and shortcomings associated with such stainless steel surgical instruments.

### SUMMARY OF THE INVENTION

The present invention is a surgical instrument having interlocking racks so that the instrument locks in a closed position, specifically when bearing a load. The interlocking racks have uniquely shaped and positioned locking teeth and a locking tab which results in an embodiment of the invention which locks in a closed position while forces are exerted against the closed instrument.

In certain embodiments of the present invention, the surgical instrument includes a first member having a handle end, a first side, a transition zone, and an arm end, the handle end defining an opening, the arm end having a tip, a second member pivotally attached to the first member, the second member having a handle end, first side, a transition zone, and an arm end, the handle end defining an opening, the arm end having a tip, a pivot pin pivotally attaches the first member and the second member. The transition zone of the first member and second member define two flutes on the first side of the first member and the first side of the second member so that the arm of each member flexes in a plane perpendicular to the tip. This embodiment of the instrument also has a rack attached to the handle end of the first member, the rack having a locking tab protruding laterally therefrom, the rack defining a plurality of engaging locking teeth. Each of the plurality of locking teeth having a locking surface that is co-planer with the locking tab and has zero rake. The second member having a complimentary rack relative to the first member.

In certain embodiments, the instrument uses a pivot pin made of nylon. In certain embodiments, the flutes defined in the transition zones have heights from about 0.1 millimeter to about 2.8 millimeters, width of from about 0.1 millimeter to about 5.0 millimeters, and the transition zone having a length of from about 50 millimeters (mm) to about 60 mm. In certain embodiments, the height and width of each of the flutes varies along the length of the transition zone. In other embodiments, the surgical instrument has tips with narrow ends and wide ends, each wide end having a diameter of about 2.0 mm, each tip having a length of from about 10 mm to about 15 mm, the tip of the first member and the tip of the second member protrude laterally from the arm end of each member, so that the arm end of each member is generally at a 90 degree angle relative to the tip, and each of the racks having a length such that contact with the opposing locking tab positions the narrow end of each tip adjacent to the wide end of the opposing tip.

Also disclosed herein is a locking device for a surgical instrument. The disclosed embodiment of the locking device includes a rack having a plurality of locking teeth, each of the plurality of locking teeth having a first side and a second side, the rack having a first end and second end, the first side of each of the plurality of locking teeth being perpendicular to an axis of a length of the rack, and a locking tab attached to the first end of the rack, the locking tab having a contact surface so that a complimentary rack and locking tab engage the first sides and second sides of the locking teeth and locking tab in order to lock the two complimentary racks together. In certain embodiments, the locking tab protrudes generally perpendicularly from the rack. In other embodiments, the contact surface of the locking tab has a length of from about 2.25 mm to about 2.75 mm and a width of from about 6.0 mm to about 6.5 mm. In certain embodiments, there are four locking teeth on each rack.

Accordingly, one aspect of the present invention is to provide a surgical instrument having flexible tips so that the tips insert into tissue along the path of least resistance.

Still another aspect of the present invention is to provide a surgical instrument having tips with wide bases so that deep penetration into tissue is limited

Another aspect of the present invention is to provide a surgical instrument having arms that flex in directions perpendicular to the closing force so that tissue damage is minimized during locking and release.

Still another aspect of the present invention is to provide a surgical instrument having a locking device to keep the surgical instrument in a closed position.

Another aspect of the present invention is to provide a locking device for a surgical instrument that keeps the surgical instrument locked in a position when bearing a load.

### BRIEF DESCRIPTION OF THE DRAWINGS

FIGS. 1A and 1B are perspective views of an embodiment of the present invention. FIG. 1A shows the embodiment in a closed position, such that the racks are contacting the opposing locking tabs. FIG. 1B shows the embodiment in a partially open position, to show the relative positioning of the opposing racks when the penetration ends of the tips are proximate.

FIG. 2 is an enlarged side view of the arm portion of the embodiment of the present invention as shown in FIG. 1.

FIGS. 3A, 3B, and 3C are cross sectional views along lines 3A-3A, 3B-3B, and 3C-3C, respectively, shown in FIG. 2 of the arm portion of the embodiment of the present invention.

FIG. 4 is a close up of the tip portion of the present embodiment of the invention as shown in FIG. 1.

FIG. 5 is a close up of the handle portion of the embodiment of the present invention which was shown in FIG. 1.

FIG. 6 is an end view of the handle portion and locking mechanism of the embodiment of the present invention as shown in FIG. 1. Shown there is the locking tab and a plurality of locking teeth located on the rack.

FIG. 7 is a perspective view of a rack showing the locking tab and plurality of locking teeth.

FIG. 8 is an end view of two racks interconnected in the locked position.

FIG. 9 is an end view of two racks in a closed, but not engaged, position, such as when unlocked and not yet in the open position.

### DETAILED DESCRIPTION OF THE INVENTION

The embodiments of the invention disclosed herein provide a locking device for use on a surgical instrument having opposing handles. The locking device includes complementary racks having locking teeth and a locking tab. The fact that at least one side of each of the locking teeth is co-planar with the locking tab is unique to the locking device. As further described herein, the interconnection of the racks locks them together. Accordingly, the handle portions of any surgical instrument to which the racks are attached are also locked in position.

Also disclosed herein is a surgical instrument constructed of a disposable material, the instrument including flexible arms, tips designed to reduce tissue damage, and the above mentioned locking device. The arms of the surgical instrument have a specific shape in order to allow flexibility in a plane that is perpendicular to the plane of force applied to close the instrument. Further, the tips of the surgical instrument are adjacent to braces to limit tissue penetration.

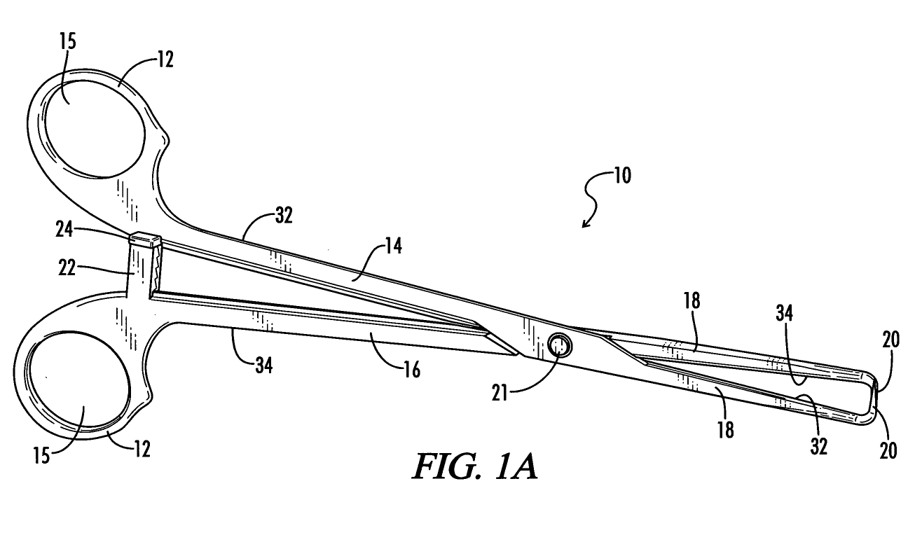

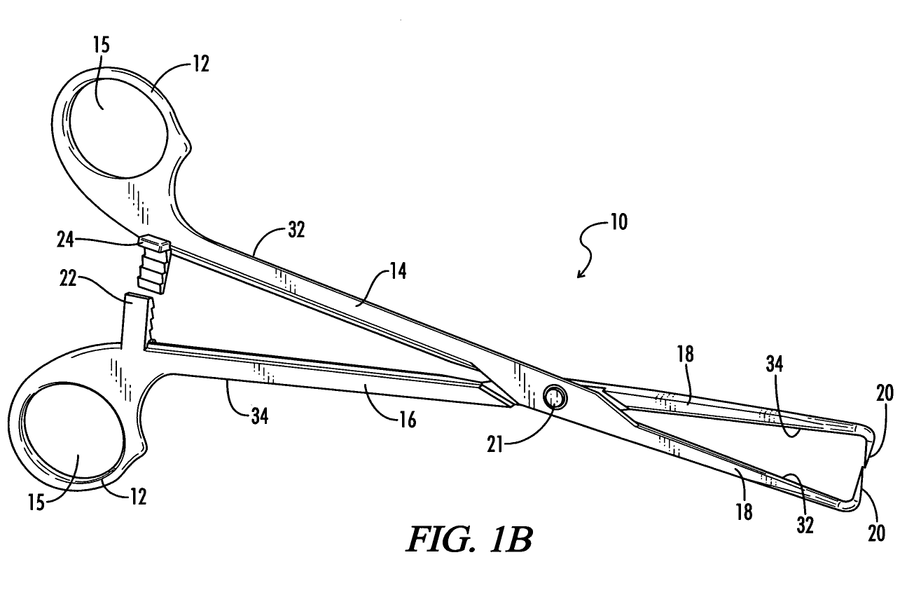

An embodiment of the surgical instrument 10 is shown in FIGS. 1A and 1B. The instrument 10 may be constructed by plastics injection molding, as is known to one of skill in the art. For example, the injection molding process maybe used, and is well known in the art. Companies that provide such services are widely commercially available. When the instrument 10 is constructed by a well known molding process, the resulting first member 14 and second member 16 are unitary, and may be pivotally attached as further described herein. Suitable material of construction of the instrument 10 includes 30% glass filled polycarbonate. As further described herein, the specific shape ofthe arms 18 are a result of testing for the desired strength and flexibility of the resulting instrument 10.

Still referring to FIGS. 1A and 1B, there is shown an embodiment of the surgical instrument 10, according to the present invention. In FIG. 1A, the instrument 10 is in the closed and locked position. In FIG. 1B, the instrument 10 is partially open. The instrument 10 includes a first member 14 and second member 16, each having a handle 12 substantially at one end, a rack 22, an arm 18, and a tip 20, substantially at the other end. The handles 12 include openings 15 to allow a user to grip the instrument 10. The first member 14 and second member 16 are connected by a pivot pin 21. The pivot pin 21 may be constructed of a nylon materials, for example Nylon 6, which weakens, or melts, when autoclaved. This is a safety feature such that the pivot pin 21 no longer properly functions and the instrument 10 may be for single use only and not require additional sterilization. As shown in the figure, the arms 18 extend on the distal side of the pivot pin 21 relative to the handles 12. The tips 20 extend generally perpendicularly from the arms 18 of each of the members 14, 16, as further described herein.

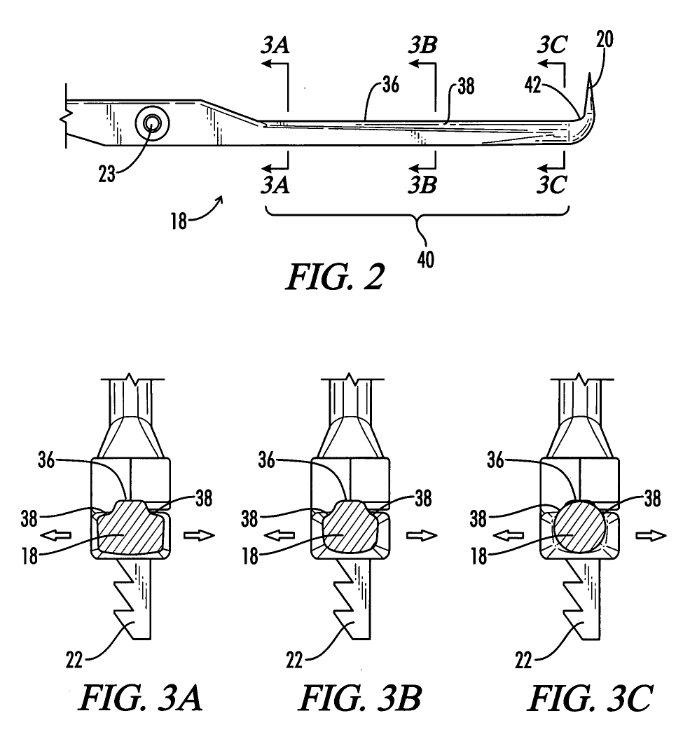

Referring now to FIG. 2, there is shown the arm 18 and tip 20 of the embodiment of the instrument 10. The pivot hole 23 is the opening into which the pivot pin 21 is inserted. With reference to the arm 18, there is a first side 36 which defines two flutes 38, as best seen in FIGS. 3A, 3B, and 3C, and described below. The section of an arm 18 in which the first side 36 defines flutes 38 is called a transition zone 40. A brace 42 connects the arm 18 and the tip 20.

Referring now to FIG. 3A, there is shown a cross sectional view along line 3A-3A, shown in FIG. 2. The cross sectional shape of the arm 18 shows the preferred shape which allows the transition zone 40 to flex so that the arm 18 moves in the directions indicated by the arrows. The flutes 38 are channels or indentions along the first side 36 of the arm 18, as shown. The first side 36 of the arm 18 is a collective description of the first side 32 or the first member 14 and the first side 34 of the second member 16. In certain embodiments, a flute 38 has a maximum cross sectional height of 2.8 mm and a maximum width of 5 mm, with the first side of the arm 36 having a height of 3.2 mm, as the flute 38 gradually phases in and out along the length of the transition zone 42, as best seen in FIGS. 2, 3A, 3B, and 3C. The preferred cross sectional shape at the end of the transition zone 40 near the pivot hole 23 is seen in FIG. 3A, which is a cross sectional view along line 3A-3A, shown in FIG. 2. Also, shown in FIG. 3C, is the cross sectional shape at the position shown along line 3C-3C, shown in FIG. 2. Accordingly, there is a change in the cross sectional shape over the 50 mm length along the arm 18.

The arms 18 are designed to pull a nine kilogram (kg) load. Again, it is the flexibility of the transition zone 40 which allows the tip 20 to penetrate tissue at the point of least resistance, such as to maneuver around and not penetrate scar tissue, which has a higher propensity to bleed. Further, in the event that the surgical instrument 10 is disturbed or removed while in the locked closed position, then the flexibility of the transition zone 40 will in part reduce tissue damage by flexing so that the tips 20 do not remain penetrated as deeply in the tissue.

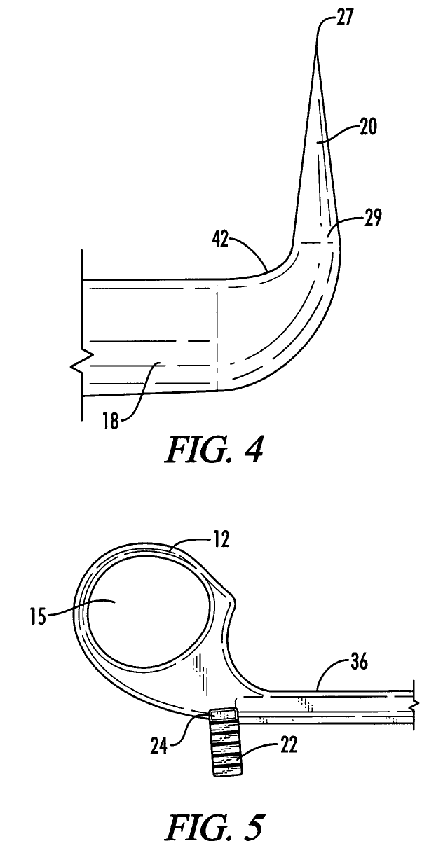

Referring now to FIG. 4, there is shown an enlargement of the brace 42 and tip 20. One aspect of the present invention is to reduce tissue damage and bleeding upon penetration of the tip 20. To that end, the tip 20 has a cone shape so that the narrow end 27 of the tip 20 penetrates tissue, while the wide end 29 of the tip 20 limits penetration due to its width. In certain embodiments, the wide end 29 of a tip 20 has a diameter of from about 2.0 mm to about 2.5 mm. In still other embodiments, the wide end 29 of a tip 20 has a diameter of 2.03 mm. Further, the brace 42 prevents further penetration due to the curved ninety degree angle. In other words, in certain embodiments, the brace 42 has an elliptical shape, with the major axis oriented along the length of the arm 18 so that the depth of penetration of the tip 20 is limited to a maximum of the length of the tip 20, and likely less penetration than that due to the extra width of the wide end 29 of the tip 20. In certain embodiments, the brace 42 may have a length of 13 mm. In certain embodiments, the brace 42 may have a cross sectional diameter of 2.03 mm. Such a length is sufficient to prevent the over-penetration of the tip 20. In certain embodiments, the arm 18, transition zone 40, brace 42, and tip 20 of the first member 14 and second member 16 should be similar, if not identical, in shape and dimensions and are symmetrically opposed as shown in FIGS. 1A and 1B.

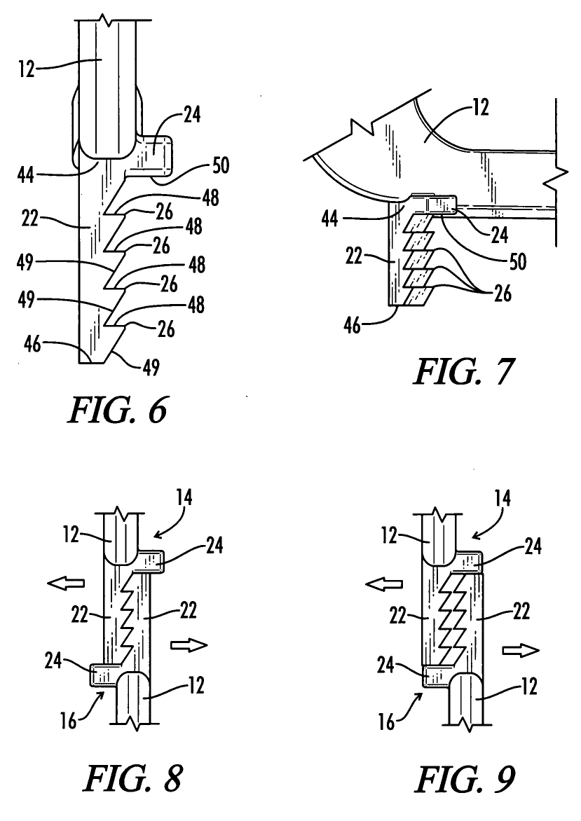

Referring now to FIG. 5, there is shown a side view of the handle 12 of the surgical instrument 10. The embodiment of the instrument 10 shown includes a locking device, which is made up of two interconnecting racks 22, best seen in FIGS. 6, 7, 8, and 9. Referring now to FIG. 6, there is shown an end view of the handle 12 showing the rack 22, locking tab 24, and plurality of locking teeth 26. In certain embodiments, a rack 22 has four locking teeth 26. In other embodiments, a rack 22 has from three to five locking teeth 26. The rack 22 has a first end 44 and a second end 46. The locking tab 24 is located on the first end 44 of the rack 22. In certain embodiments, the locking tab 24 protrudes laterally from the handle 12 a distance of 2.3 mm. In other embodiments, the locking tab 24 protrudes laterally from the handle 12 a distance of from 10 mm to 20 mm, in order to ensure that it contacts the opposing rack.

As seen in FIGS. 6, 7, 8, and 9, each of the plurality of locking teeth 26 has a locking surface 48, which is co-planar with the locking tab 24. As used herein, the locking surface 48 may also be called the first side of the locking tooth 26. The second side 49 of each locking tooth 26 is also shown in FIG. 6. It is further important that each locking surface 48 has zero rake, that is, it is perpendicular to the force being applied to open or close the surgical instrument 10. Thus, the engaging locking teeth 26 and locking tabs 24 create a single closed and locked position, as shown in FIG. 8. While in a locked position, in order to release the locked instrument 10, it is not necessary to reverse the handles 12. Rather, unlocking is accomplished by pulling the handles in opposite directions relative to the engaged racks 22, as shown by the arrows in FIG. 8. When the handles are moved in opposite directions, as best seen in FIG. 9, the locking teeth 26 are not locked and the instrument 10 may open, as seen in FIG. 1B. In use, force is applied on the handles 12 of the surgical instrument 10 so that the second end 46 of the rack 22 of the first member 14 contacts the contact surface 50 of the locking tab 24 of the second member 16. At that point, lateral force is applied, in the direction opposite of the arrows shown in FIG. 8 in order to engage the locking teeth 26 of each rack 22. In certain embodiments, the tips 20 or arms 18 are bearing a load in order to allow the locking teeth 26 to remain engaged after the lateral force has been applied and is no longer present. In certain embodiments, the load bearing force on the tips 20 or arms is at least 2 kg. In certain embodiments, when the instrument 10 is in the closed position, i.e. the second end 46 of one rack contacting the contact surface 50 of the other rack 22, the narrow end 27 of one tip 20 is adjacent to the wide end 29 of the opposing tip 20. Such proximity allows for gripping or manipulating tissue and locking the instrument 10 while doing so.

In an alternate embodiment, the locking device maybe attached to, or applied to, any instrument, including specifically any surgical instrument. If such instrument is injection molded, then the locking device, being made up of two racks 22 having locking teeth 26 and locking tab 24, as described above, may be molded into an appropriate position on the instrument to perform the locking function. Alternately, the racks 22 may be fastened to, or fixed to, an instrument as known to one of ordinary skill in the art when the instrument is not injection molded, or if the instrument is preexisting. Based upon the positioning of the locking device, such instrument may be locked in a closed position, or other configuration. In an alternate embodiment, the locking device including the engaging opposing racks 22, as described herein, may be attached to another medical device in which locking such device in a closed position is desirable.

All references, publications and patents disclosed herein are expressly incorporated by reference.

Thus, it is seen that the apparatus of the present invention readily achieves the ends and advantages mentioned as well as those inherent therein. While certain preferred embodiments of the invention have been illustrated and described for purposes of the present disclosure, numerous changes in the arrangement of parts or steps of the methods may be made by those skilled in the art, which changes are encompassed within the scope and spirit of the present invention, as defined by the following claims.

What is claimed is:

1. A surgical instrument, comprising:
a first member having a handle end, a first side, a transition zone, and an arm end, the handle end defining an opening, the arm end having a tip;
a second member pivotally attached to the first member, the second member having a handle end, first side, a transition zone, and an arm end, the handle end defining an opening, the arm end having a tip;
a pivot pin pivotally attaching the first member and the second member;
wherein the transition zone defines two flutes on the first side of the first member so that the arm flexes in a plane perpendicular to the tip;
wherein the transition zone defines two flutes on the first side of the second member so that the arm flexes in a plane perpendicular to the tip;
a rack attached to the handle end of the first member, the rack having a locking tab protruding laterally therefrom, wherein the rack defines a plurality of locking teeth, each of the plurality of locking teeth having a locking surface that is parallel to the locking tab;
a rack attached to the handle end of the second member, the rack having a locking tab protruding laterally therefrom, wherein the rack defines a plurality of locking teeth, each of the plurality of locking teeth having a locking surface that is parallel to the locking tab.

2. The surgical instrument of claim 1, wherein the pivot pin is constructed of a nylon.

3. The surgical instrument of claim 2, wherein each of the flutes defined in the transition zone of the first member and the transition zone of the second member has a height of from about 0.1 mm to about 2.8 mm and a width of from about 0.1 mm to about 5.0 mm.

4. The surgical instrument of claim 3, wherein the transition zone of the first member and the transition zone of the second member define the flutes along the length of the transition zone for from about 50 mm to about 60 mm.

5. The surgical instrument of claim 4, wherein the height and the width of each of the flutes varies along the length of the transition zone.

6. The surgical instrument of claim 5, further comprising each tip having a narrow end and a wide end, the wide end having a diameter of about 2.0 mm.

7. The surgical instrument of claim 6, wherein each tip having a length of from about 10 mm to about 15 mm.

8. The surgical instrument of claim 7, wherein the tip of the first member and the tip of the second member protrude laterally from the arm end of each member, so that the arm end of each member is generally at a 90 degree angle relative to the tip.

9. The surgical instrument of claim 8, wherein each of the racks has a length such that contact with the opposing locking tab positions the narrow end of each tip adjacent to the wide end of the opposing tip.

10. A locking device for a surgical instrument, comprising:
a rack, defining therein a plurality of locking teeth, each of the plurality of locking teeth having a first side and a second side, the rack having a first end and a second end;
wherein the first side of each of the plurality of locking teeth is perpendicular to an axis of a length of the rack;
a locking tab attached to the first end of the rack, the locking tab having a contact surface, so that a complementary rack and locking tab engage the first sides and second sides of the locking teeth and contact surface of the locking tab in order to lock the two complementary racks together.

11. The locking device of claim 10, wherein the locking tab protrudes generally perpendicularly from the rack.

12. The locking device of claim 11, wherein the contact surface of the locking tab has a length of from about 2.25 mm to about 2.75 mm, and a width of from about 6.0 mm to about 6.5 mm.

13. The locking device of claim 12, wherein the plurality of locking teeth is four locking teeth.

14. A surgical instrument having a locking device, comprising:
a first member having a handle end, a first side, a transition zone, and an arm end, the handle end defining an opening, the arm end having a tip;
a second member pivotally attached to the first member, the second member having a handle end, first side, a transition zone, and an arm end, the handle end defining an opening, the arm end having a tip;
a pivot pin pivotally attaching the first member and the second member, wherein the pivot pin is constructed of a material that melts at temperatures above 260 degrees Celsius;
wherein the transition zone defines two flutes on the first side of the first member so that the arm flexes in a plane perpendicular to the tip;
wherein the transition zone defines two flutes on the first side of the second member so that the arm flexes in a plane perpendicular to the tip;
a rack attached to the handle end of the first member, the rack having a locking tab protruding laterally therefrom, wherein the rack defines a plurality of locking teeth, each of the plurality of locking teeth having a locking surface that is parallel to the locking tab;
a rack attached to the handle end of the second member, the rack having a locking tab protruding laterally therefrom, wherein the rack defines a plurality of locking teeth, each of the plurality of locking teeth having a locking surface that is parallel to the locking tab;
wherein each of the flutes defined in the transition zone of the first member and the transition zone of the second member has a height of from about 0.1 mm to about 2.8 mm and a width of from about 0.1 mm to about 5.0 mm;
wherein the height and the width of each of the flutes varies along the length of the transition zone;
wherein the tip of the first member and the tip of the second member each have a narrow end and a wide end, the wide end having a diameter of about 2.0 mm;
wherein the tip of the first member and the tip of the second member protrude inwardly from the arm end of each member, so that the arm end of each member is generally at a 90 degree angle to each of the tips;
wherein the locking tab has a length of from about 2.25 mm to about 2.75 mm, and a width of from about 6.0 mm to about 6.5 mm.
